(19) United States
(12) Patent Application Publication
(10) Pub. No.: US 2026/0325763 A1
(43) Pub. Date: Oct. 22, 2026

(54) A SUBASSEMBLY OF A MEDICAMENT DELIVERY DEVICE AND A MEDICAMENT DELIVERY DEVICE

(71) Applicant: SHL Medical AG, Zug (CH)

(72) Inventors: Daniel Carlsson, Nacka Strand (SE); Antonio Farieta, Zug (CH)

(21) Appl. No.: 19/104,760

(22) PCT Filed: Aug. 8, 2023
(86) PCT No.: PCT/EP2023/071877

§ 371 Date: Feb. 19, 2025

(30) Foreign Application Priority Data
Aug. 22, 2022 (EP) 22191402.1

(51) Int. Cl. A61M 5/32; A61M 5/20

(52) U.S. Cl. CPC A61M 5/3204; A61M 5/20

(57) ABSTRACT

The present disclosure generally relates to medicament delivery devices such as autoinjectors, and particularly concerns a subassembly for removing a needle shield.

### CROSS-REFERENCE TO RELATED APPLICATIONS

The present application is a U.S. National Phase Application pursuant to 35 U.S.C. § 371 of International Application No. PCT/EP 2023/071877 filed Aug. 8, 2023, which claims priority to EP Patent Application No. 22191402.1 filed Aug. 22, 2022. The entire disclosure contents of these applications are herewith incorporated by reference into the present application.

### TECHNICAL FIELD

The present disclosure generally relates to medicament delivery devices such as autoinjectors, and particularly concerns a needle cover and subassembly for removing a needle shield.

### BACKGROUND

A number of medical conditions require injections. These days, a number of different injection devices exist, including various types of pen injectors, autoinjectors and on-body devices. Although many of these devices have enabled major improvements in the management of a number of medical conditions, various limitations do still exist in the current technology. Not least amongst these are the difficulties faced by patients that require frequent injections and by patients that need to inject particularly viscous drugs. In considering these problems, the applicant has appreciated that various developments could be made to help improve the medicament delivery devices on the market today, for example concerning removal of a needle shield of the medicament delivery devices, which are set out in more detail below.

### SUMMARY

An object of the present disclosure is to provide a needle cover and a subassembly for a medicament delivery device which solves, or at least mitigates problems of the prior art.

According to a first aspect of the present disclosure, there is provided a subassembly of a medicament delivery device for removing a needle shield of the medicament delivery device extending from a proximal end to a distal end along a longitudinal axis, the needle shield is configured to cover a needle attached to a proximal end of a medicament container accommodated in a housing, the subassembly comprising: a cap configured to cover the needle shield, a flexible gripper attached to the cap, the flexible gripper extending distally towards the medicament container, the gripper is configured to be arranged radially outside the needle shield, a syringe carrier adapted to be accommodated in the housing, the syringe carrier comprising a tubular proximal body configured to receive the medicament container radially inside the tubular proximal body, the tubular proximal body comprises a first portion and a second portion proximally adjacent to the first portion, wherein the tubular proximal body comprises a first portion and a second portion proximally adjacent to the first portion, wherein the first portion comprises either a first inner diameter larger than an outer diameter of the flexible gripper, or a cut-out; wherein the second portion comprises a second inner diameter smaller than the outer diameter of the flexible gripper,

Embodiments of the present disclosure advantageously provides for removal of the removable needle shield when the cap is moved proximally and also provides efficient assembling process. The embodiments as disclosed provided that the flexible gripper is able to flex radially outward when it lines up with the first portion or the cut-out of the tubular proximal body. Therefore, during the assembling process, the needle shield can easily be moved past the flexible gripper and enter into the cap. When the device is assembled, the motion of the cap in the proximal direction is typically in preparation for using the medicament delivery device. Advantageously, the removal of the cap causes removal of the needle shield by means of the flexible gripper so that the user does not have to manually remove the needle shield which may cause unnecessary risk of sharp injury.

The first portion and the second portion of the tubular proximal body may be coaxial or circumferentially offset.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the components thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, “circumferentially” refer to a circumference or a circumferential direction 301 relative to an axis 102, typically a central axis extending in the direction of the longest extension of the device and/or component. Similarly, “radial” or “radially” refer to a direction 302 extending radially relative to the axis, and “rotation”, “rotational” and “rotationally” refer to rotation relative to the axis.

According to one embodiment, the flexible gripper is received inside the tubular proximal body of the syringe carrier. For example, when the tubular proximal body of the syringe carrier is generally cylindrical.

Alternatively, in another embodiment, a cut-out is arranged in a wall of the tubular proximal body of the syringe carrier. The cut-out may act as a window for the user to observe the contained medicament. In this example, the flexible gripper can be lined up with the cut-out before being moved to be aligned with the second portion of the tubular proximal body of the syringe carrier. In this example, the flexible gripper may be positioned within the cut-out or radially extend out from the cut-out. In this embodiment, the cut-out defines the first portion of the tubular proximal body.

According to one embodiment, the gripper may comprise a radially outwards extending protrusion. The radially outwards extending protrusion provides for an advantageous way to realize engagement between the tubular proximal body and the flexible gripper, especially for engagement with the second portion of the tubular proximal body that causes the flexible gripper to be pressed radially inwards.

According to one embodiment, the flexible gripper may be annularly shaped.

According to one embodiment, the flexible gripper may comprise at least one distally extending flexible arm. The distally extending flexible arms provide a simple yet robust way of providing the radially inwards flexing motion of the flexible gripper.

According to one embodiment, each flexible arm comprises a radially outwards extending protrusion.

According to one embodiment, the flexible gripper engages with a distal end portion of the needle shield. The engagement provides for removal of the needle shield as the cap moves proximally.

According to one embodiment, wherein the gripper engages with a side surface of the needle shield. The engagement provides for removal of the needle shield as the cap moves proximally.

According to one embodiment, wherein the flexible gripper may be configured to engage with the needle shield by a frictional connection.

According to one embodiment, the flexible gripper may comprise a radially inwards extending protrusion configured to abut against a distal end of the needle shield when the gripper moves from the first portion towards the second portion of the tubular proximal body. This form-fit engagement provides for a robust engagement between the flexible gripper and the needle shield.

According to one embodiment, the flexible gripper may comprise a tubular body for receiving the needle shield, the tubular body comprising at least one protrusion extending radially inwards to engage with the needle shield by friction.

According to one embodiment, the at least one protrusion of the tubular body is at least one rib extending longitudinally along the cap.

According to one embodiment, an inner diameter of the tubular body of the flexible gripper may be larger than an outer diameter of the needle shield.

According to one embodiment, the gripper and the cap may be made in one piece a single component.

According to one embodiment, the gripper may be made of a plastic material.

There is further provided a medicament delivery device comprising the subassembly of any of the herein disclosed embodiments.

There is further provided a method of assembling of a medicament delivery device. The method comprises the following steps in the following order:
providing a subassembly, wherein the subassembly comprises a cap comprising a distally extending flexible gripper; and a syringe carrier comprising a tubular proximal body, wherein the tubular proximal body comprises a first portion and a second portion proximally adjacent to the first portion, wherein the first portion comprises at least one of a first inner diameter larger than an outer diameter of the flexible gripper; wherein the second portion comprises a second inner diameter smaller than the outer diameter of the flexible gripper; and wherein the flexible gripper is received within the first portion of the proximal tubular body;
providing a medicament container assembly; wherein the medicament container assembly comprises a medicament container and a needle shield; wherein the medicament container comprises a needle attached at the proximal end of the medicament container; and wherein the needle is covered by the needle shield;
inserting the medicament container assembly into the tubular proximal body of the syringe carrier from a distal end of the tubular end of the proximal tubular body of the syringe carrier; and
axially moving the medicament container assembly within the tubular proximal body until the flexible gripper is either radially aligned with the needle shield or distally positioned relative to the needle shield.

Generally, all terms used in the claims are to be interpreted according to their ordinary meaning in the technical field, unless explicitly defined otherwise herein. All references to “a/an/the member, apparatus, component, means, etc.” are to be interpreted openly as referring to at least one instance of the member, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1 is a perspective view of an autoinjector according to embodiments of the present disclosure;

FIG. 2 is an exploded view of an autoinjector according to embodiments of the present disclosure;

FIG. 3 is a perspective view of a cap according to embodiments of the present disclosure;

FIG. 4 is another perspective view of the cap according to embodiments of the present disclosure;

FIG. 5 is a perspective view of a syringe holder according to embodiments of the present disclosure;

FIG. 6 is a close up perspective view of the syringe holder according to embodiments of the present disclosure;

FIG. 7 is a perspective view of a removable needle shield according to embodiments of the present disclosure;

FIG. 8 is a cross-section of a subassembly in an initial assembled state according to embodiments of the present disclosure;

FIG. 9 is a cross-section of a subassembly when the cap is partly removed according to embodiments of the present disclosure; and

FIG. 10 is a cross-section of a close up of medicament delivery device at the distal end of the syringe according to embodiments of the present disclosure.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like members throughout the description.

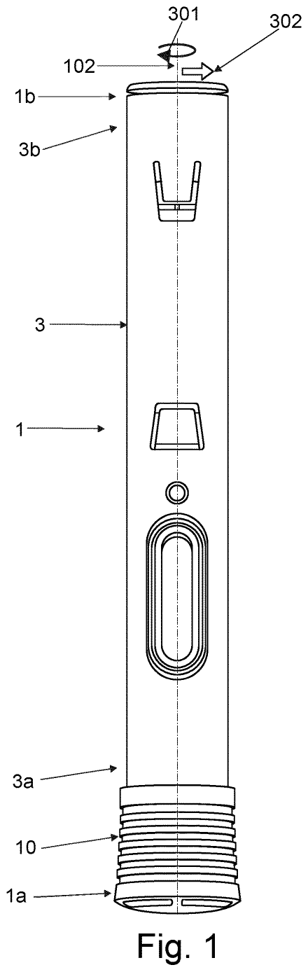

FIG. 1 shows an example of a medicament delivery device 1 such as an autoinjector according to embodiments of the present disclosure. The medicament delivery device 1 is configured to expel medicament from a medicament container via a medicament delivery member such as a needle, to a user at a dose delivery site. The medicament delivery device 1 extends from a proximal end la to a distal end 1b relative to the axis 102.

The medicament delivery device 1 comprises a housing 3 which has a proximal end 3a and a distal end 3b.

The medicament delivery device 1 further comprises a cap 10 releasably attached to the proximal end 3a of the housing 3.

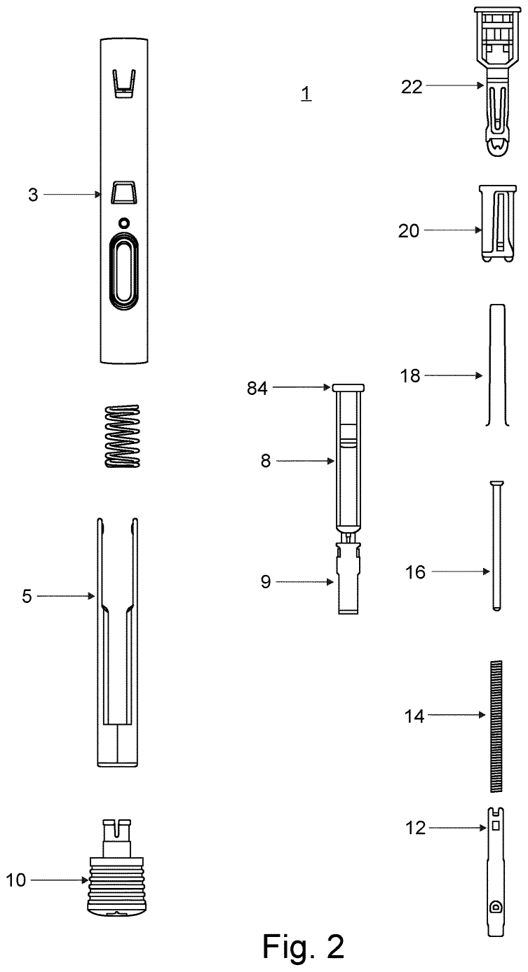

FIG. 2 is an exploded view of the medicament delivery device 1 which further comprises a needle cover 5, a needle cover spring 7, a medicament container 8, i.e., a syringe, accommodated in the housing 3, and a needle shield 9 configured to cover a needle attached to a proximal end of the medicament container 8.

The medicament delivery device 1 further comprises a plunger rod 12, a plunger rod spring 14, a plunger spring guide rod 16, a U-bracket 18, a rotator 20, and a rear cap 22. Generally, the plunger rod 12 is configured to move a stopper in the syringe 8 in the proximal direction upon being moved by the plunger rod spring 14, so that a medicament contained in the medicament container 8 is expelled.

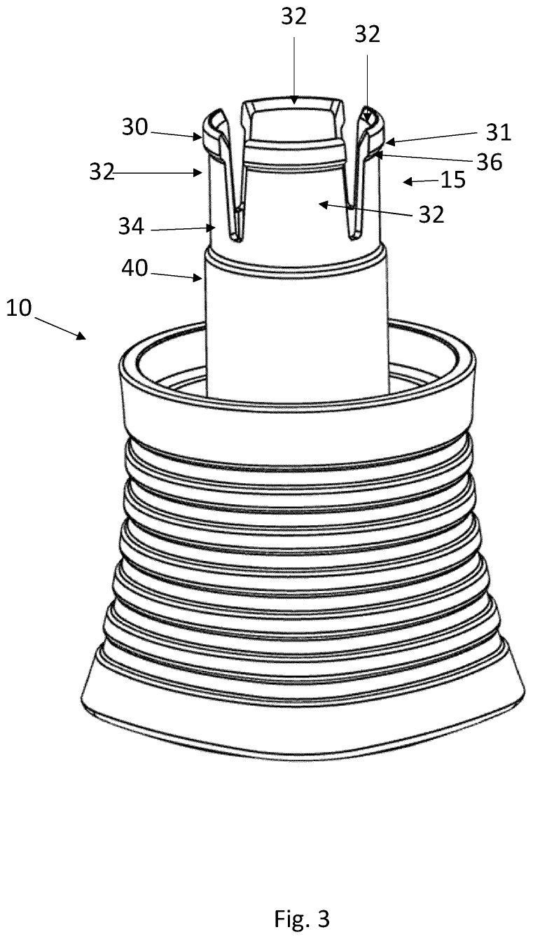

FIG. 3 is a perspective view of a cap 10 according to embodiments of the present disclosure. A flexible gripper 15 is attached to the cap 10 and extends distally towards the medicament container 8 shown in FIG. 2. Further, the flexible gripper 15 is configured to be arranged radially outside the needle shield 9.

The flexible gripper 15 comprises a set of distally extending flexible arms 32. Each flexible arm 32 comprises a radially outwards extending protrusion 30 on their respective distal end 31. The distal end 31 is the free end of the flexible arm 32 that can radially flex relative a fixed end 34 in the proximal end of the flexible arm 32. The fixed proximal end 34 is attached to or made in the same piece as a tubular body 40 of the cap.

The flexible gripper 15 is annularly shaped and has a circular circumference. In this embodiment, the annular flexible gripper 15 comprises four flexible arms 32 symmetrically arranged and extending in the distal direction away from the tubular body 40. Indeed, is possible that the flexible gripper comprises at least two flexible arms.

The tubular body 40 has an outer diameter that is larger than the outer diameter of the flexible gripper 15 at the fixed ends 34 of the flexible arms 32.

The radially outwards extending protrusions 30 includes a proximally facing surface 36 that is inclined with respect to the axis 102.

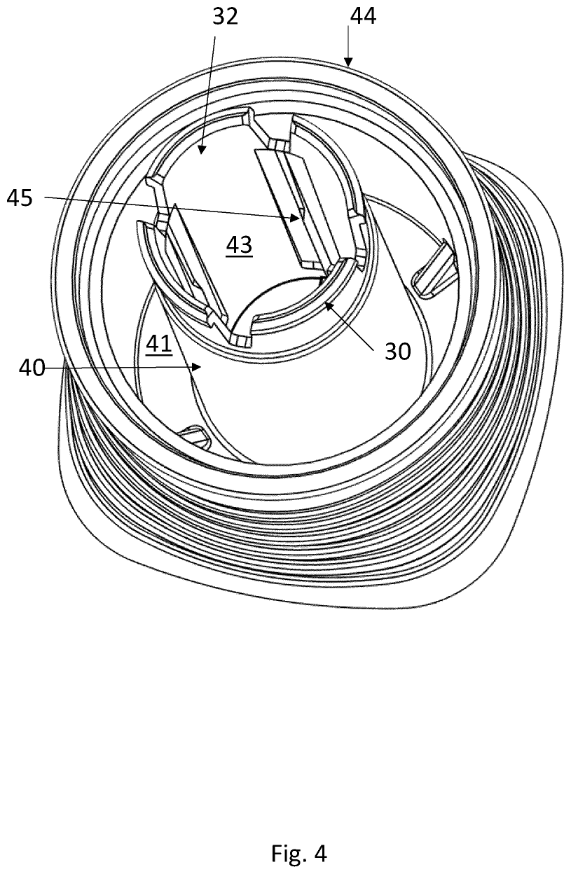

FIG. 4 is another perspective view of the cap 10 more clearly illustrating the inner space of the tubular body 40. The tubular body is attached to a distally facing surface 41 at the proximal end of the cap 10. Further, an outer annular wall 44 is coaxially arranged with the tubular body 40 and is adapted to be grabbed by a user for removal of the cap 10 from the medicament delivery device 1.

The inner space of the tubular body 40 is adapted to receive the needle shield 9. Thus, the inner diameter of the tubular body 40 is larger than the outer diameter of the needle shield 9. However, the tubular body 40 comprises at least one protrusion 45 extending radially inwards to engage with the needle shield by friction. Preferably, the tubular body comprises a set of protrusions 45 symmetrically arranged on a side wall 43 of the tubular body 40 facing radially inwards.

In this embodiment, the at least one protrusion 45 of the tubular body 40 is at least one rib, or crush-rib, extending longitudinally along the cap 10. The rib 45 may extend along for example about half the longitudinal length of the tubular body 40, or about 75% or more of the longitudinal length of the tubular body 40. In some embodiments the rib extend about 20% or more of the longitudinal length of the tubular body 40.

Preferably, the flexible gripper 15 and the cap 10 is made in one piece as a single component. The flexible gripper 15 may for example be made of a plastic material.

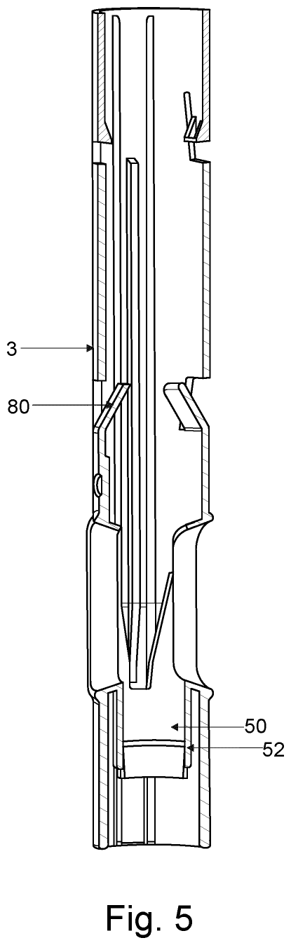

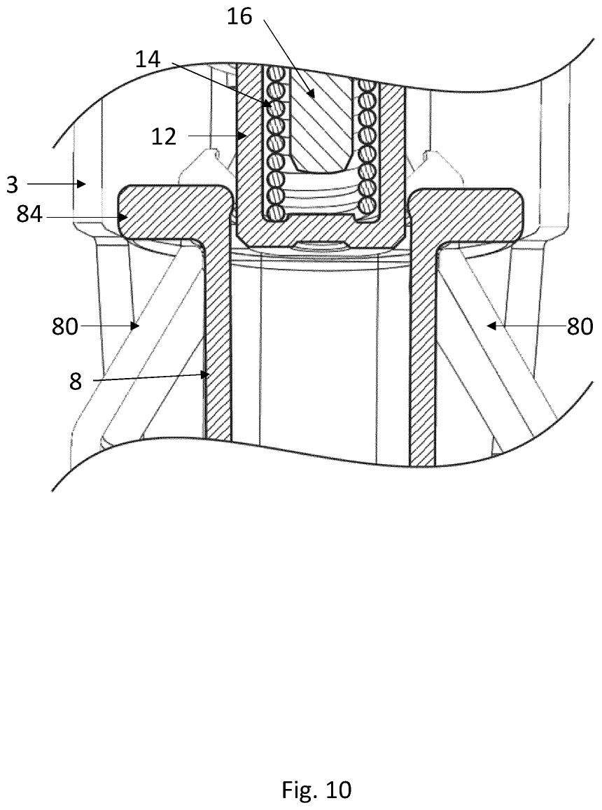

FIG. 5 is a cross-sectional view of a syringe carrier 50. The syringe carrier 50 is accommodated in the housing 3 and may or may not be made in one piece with the housing 3 depending on the specific implementation at hand. Flexible tabs 80 are configured to abut against a flange 84 of the syringe 8 as will be discussed in more detail with reference to FIG. 10.

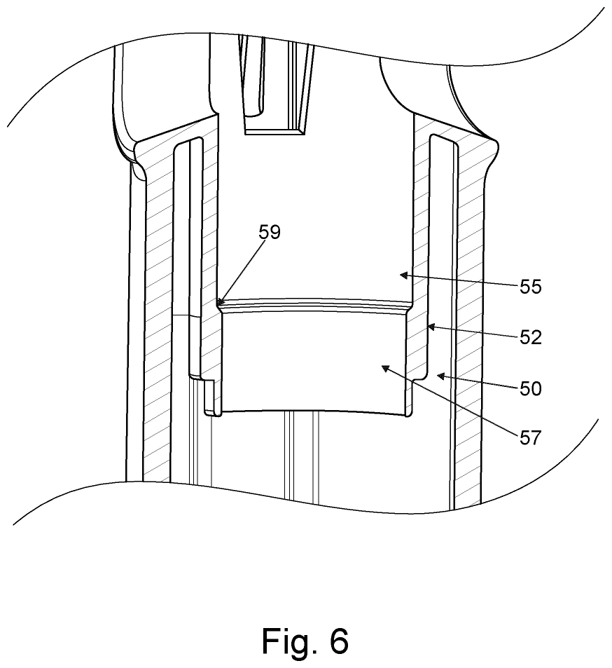

As is better seen in FIG. 6, the syringe carrier 50 comprises a tubular proximal body 52. The tubular proximal body 52 is configured to receive the medicament container 8 radially inside the tubular proximal body 52. In other words, the medicament container 8 is fitted inside the tubular proximal body 52 such that the needle is fed through the tubular proximal body 52.

The tubular proximal body 52 comprises a first portion 55 and a second portion 57 proximally adjacent to the first portion 55. In the depicted embodiment, the first portion 55 of the tubular proximal body 52 has a first inner diameter larger than an outer diameter of the flexible gripper 15. However, in other possible embodiments first portion 55 may instead include cut-outs through which the protrusion 30 of the flexible arms 32 are arranged.

The second portion 57 of the tubular proximal body 50 has a second inner diameter smaller than the outer diameter of the flexible gripper 15. The tubular body 50 further includes a distally facing surface 59 that is inclined with respect to the longitudinal axis 102. The inclined surface is at the interface between the first portion 55 and the second portion 57.

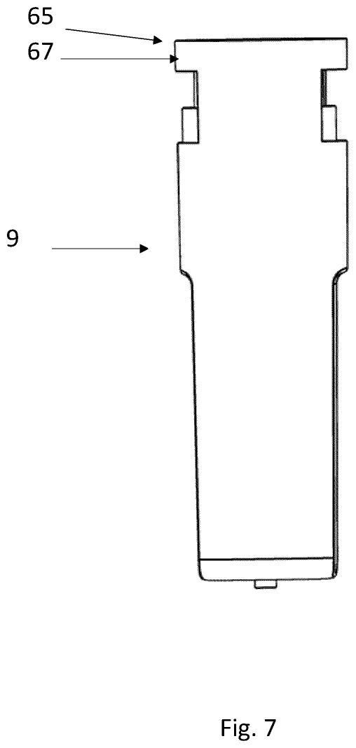

FIG. 7 is a perspective view of a needle shield 9. The needle shield 9 may be a rigid needle shield (RNS) or a flexible needle shield (FNS). The needle shield 9 comprises a distal end portion 65 that is proximally located with respect to the distal end of the flexible gripper 31. The flexible gripper 15 is configured to engage with the distal end portion 65 of the needle shield and/or the side surface 67 of the needle shield. For example, the flexible gripper 15 may comprise a radially inwards extending protrusion configured to abut against the distal end 65 of the needle shield when the flexible gripper moves from the first portion towards the second portion of the tubular proximal body.

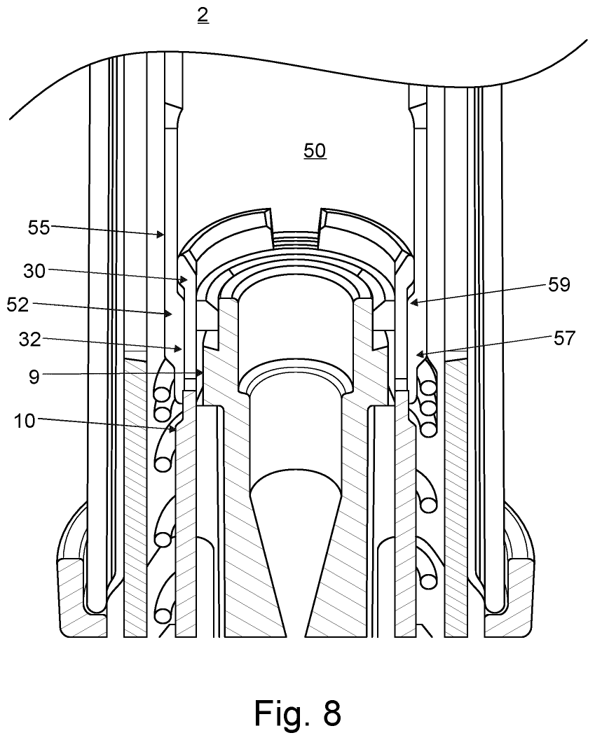

FIG. 8 is a cross-section of a subassembly 2 comprising the cap 10 and the syringe carrier 50. In FIG. 8, the subassembly 2 is in an initial assembled state. The flexible gripper 15, or more precisely the protrusions 30 in this embodiment are located at the first portion 55 of the tubular proximal body 52. In this state the flexible arms 32 are in a neutral position, i.e., not flexed inwards, and has therefore not engaged with the needle shield 9.

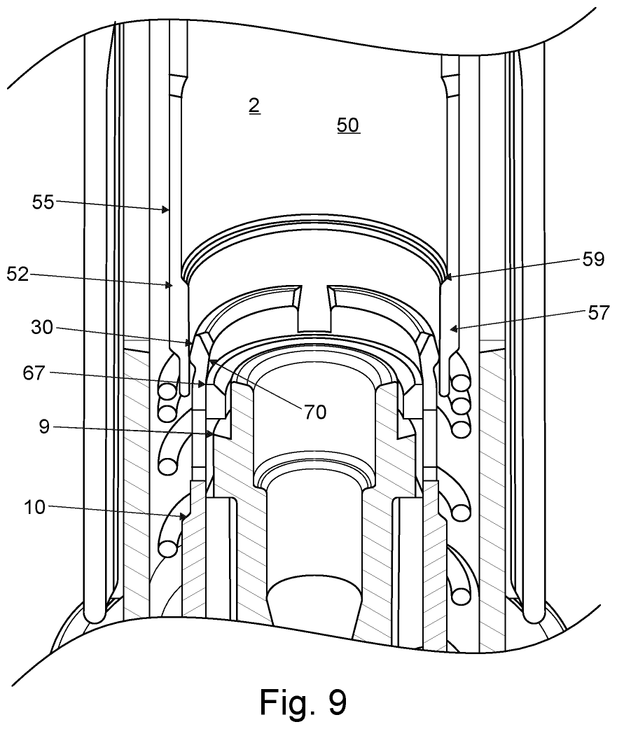

Turning to FIG. 9, where the subassembly is shown in a state with the cap 10 being moved in the proximal direction for removal. The flexible gripper 15, here the flexible arms 32 and the protrusions 30 have moved from the first portion 55 to the second portion 57 of the tubular proximal body 52. As the flexible arms reach the second portion 57 having inner diameter smaller than the outer diameter of the flexible gripper 15 at the protrusion 30, the flexible arms 32 are pressed radially inwards to engage with the needle shield 9. Here a radially inwards facing surface 70 of the flexible arms 32 engages with the side surface 67 of the needle shield by friction.

The ribs 45 of the cap 10 discussed in relation to FIG. 4 strengthens the connection between the cap 10 and the needle shield 9 and further prevents the needle shield 9 from falling out from the cap after removal of the cap from the medicament delivery device 1.

FIG. 10 is a cross-section of a medicament delivery device 1. Here the distal end of the syringe 8 is shown having a distal annular flange 84. In order to provide a support for the syringe 8 that maintains the syringe 8 in place when the cap 10 pulls the needle shield 8 off the syringe 8, the flange is supported on the flexible tabs 80 that reach radially inwards so that a distal end of the flexible tabs contact a proximally facing surface 86.

A medicament delivery device (such as an autoinjector) may generally include various other components. For example, a sensor unit which may recognize injection events, such as the autoinjector inserted into an attachment portion of e.g., a pad, injection started, and injection ends, a memory unit which is configured to store the recorded data during the injection, a connectivity unit configured to transmit the stored data to a smart device or the network directly, a processing unit configured to control the entire system and processes the data before transmitting it, and/or user interface units that are configured to provide feedback to the patient, such as status LEDs, haptic, and/or audio feedback.

When the medicament delivery device is placed into the attachment portion, the sensors inside of the pad are configured to recognize the event and give feedback to the patient via haptic/visual or audio elements.

When the injection finishes, the sensors are configured to recognize the event and give feedback to the patient again. Further, the collected data is stored in the memory unit and may be transmitted to the smart device/network via the connectivity unit after the injection event finishes.

The sensor can be one of or the combination of the following: a mechanical switch, a Hall-effect sensor, an accelerometer.

The mechanical switch, hall-effect sensor, or accelerometer can be used for detection of the insertion of the auto-injector into an injection port.

The accelerometer can be used for detecting injection events.

Possible wireless communication methods include Bluetooth and Cellular Networks.

Bluetooth connectivity requires a smart device to transmit the stored data to the network and it requires a pairing action between the pad and the smart device before being able to use the supporting pad in case of 2-way connection. But it's a cheaper alternative and it requires less space on PCB. A 1-y connection does not require pairing.

The cellular network does not require any pairing process, it can be used as a plug-n-play device, no prior setup is needed, but it's more expensive and it requires more space on PCB.

Depending on the requirements of the product any of those two technologies can be used.

Such processing units may comprise a logic circuit or control unit including a microprocessor, microcontroller, programmable digital signal processor or another programmable device. The processing circuitry may also, or instead, each include an application specific integrated circuit, a programmable gate array or programmable array logic, a programmable logic device, or a digital signal processor. Where the processing circuitry includes a programmable device such as the microprocessor, microcontroller or programmable digital signal processor mentioned above, the processor may further include computer executable code that controls operation of the programmable device.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, antibodies, proteins, fusion proteins, peptibodies, polypeptides, pegylated proteins, protein fragments, protein analogues, protein variants, protein precursors, and/or protein derivatives. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-la (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

The inventive concept has mainly been described above with reference to a few examples. However, as is readily appreciated by a person skilled in the art, other embodiments than the ones disclosed above are equally possible within the scope of the inventive concept, as defined by the appended claims.

1-15. (canceled)

16. A subassembly of a medicament delivery device for removing a needle shield of the medicament delivery device extending from a proximal end to a distal end along a longitudinal axis, the needle shield is configured to cover a needle attached to a proximal end of a medicament container accommodated in a housing, the subassembly comprising:
a cap configured to cover the needle shield;
a flexible gripper attached to the cap, the flexible gripper extending distally towards the medicament container, the flexible gripper is configured to be arranged radially outside the needle shield; and
a syringe carrier adapted to be accommodated in the housing, the syringe carrier comprising a tubular proximal body configured to receive the medicament container radially inside the tubular proximal body,
wherein the tubular proximal body comprises a first portion and a second portion proximally adjacent to the first portion, wherein the first portion comprises either a first inner diameter larger than an outer diameter of the flexible gripper, or a cut-out, wherein the second portion comprises a second inner diameter smaller than the outer diameter of the flexible gripper,
wherein when the cap is moved proximally for removal from the medicament delivery device, the flexible gripper moves from the first portion towards the second portion of the tubular proximal body, whereby the flexible gripper is pressed radially inwards to engage with the needle shield.

17. The subassembly according to claim 16, wherein the flexible gripper comprises a radially outwards extending protrusion.

18. The subassembly according to claim 16, wherein the flexible gripper is annularly shaped.

19. The subassembly according to claim 16, wherein the flexible gripper comprises at least one distally extending flexible arm.

20. The subassembly according to claim 19, wherein each flexible arm comprises a radially outwards extending protrusion.

21. The subassembly according to claim 16, wherein the flexible gripper engages with a distal end portion of the needle shield.

22. The subassembly according to claim 16, wherein the flexible gripper engages with a side surface of the needle shield.

23. The subassembly according to claim 16, wherein the flexible gripper is configured to engage with the needle shield by a frictional connection.

24. The subassembly according to claim 16, wherein the flexible gripper comprises a radially inwards extending protrusion configured to abut against a distal end of the needle shield when the flexible gripper moves from the first portion towards the second portion of the tubular proximal body.

25. The subassembly according to claim 16, wherein the flexible gripper comprises a tubular body for receiving the needle shield, the tubular body comprising at least one protrusion extending radially inwards to engage with the needle shield by friction.

26. The subassembly according to claim 25, wherein the at least one protrusion of the tubular body is at least one rib extending longitudinally along the cap.

27. The subassembly according to claim 25, wherein an inner diameter of the tubular body of the flexible gripper is larger than an outer diameter of the needle shield.

28. The subassembly according to claim 16, wherein the flexible gripper and the cap is made in one piece as a single component.

29. The subassembly according to claim 16, wherein the flexible gripper is made of a plastic material.

30. A medicament delivery device comprising the subassembly according to claim 16.
